(19) United States
(12) Patent Application Publication
(10) Pub. No.: US 2007/0125874 A1
(43) Pub. Date: Jun. 7, 2007

(54) Aromatic dispenser container

(76) Inventor: Jan C. Alexander, Corpus Christi, TX (US)

Correspondence Address: ALBERT B. KIMBALL, JR.;BRACEWELL & PATTERSON L L P, 711 LOUISANA SUITE 2300, HOUSTON, TX, 77002, US

(21) Appl. No.: 11/295,932

(22) Filed: Dec. 7, 2005

(51) Int. Cl. A61L 9/04; A24F 25/00

(52) U.S. Cl. 239/44; 239/45; 239/43

(57) ABSTRACT

An aromatic dispenser/container dispenses a scent or aroma, based on a fluid in the dispenser. The fluid containing the scented aroma is contained within a bottle or jar, typically of some decorative configuration. The fluid need not be flammable and no flame or heat source is required. A top of the bottle or jar has a top or cap member attached to form a closed container for the scented fluid. The cap member is formed of a porous, clay-type material of the type used in forming porcelain articles. The clay material of the cap member is formed during a first firing of the material is known as bisque, or biscuit. The bisque material of the cap member is hardened and more rigid than are original clay material, but is yet still permeable and porous enough to allow the vapors from the fluid in the container to permeate from the bottle through the bisque material and into the nearby region. The clay material can also be shaped before first firing to form it into bisque into a variety of ornamental or aesthetically pleasing shapes or figures to give the dispenser a decorative appearance.

### BACKGROUND OF THE INVENTION

1. Field of the Invention

The present invention relates to a dispenser container for an aromatic or scented fluid which dispenses or releases the fluid as a vapor in the area of the container without requiring a flame or other heat source.

2. Description of the Related Art

There have been a number of dispensers in which an aromatic or scented fluid in a container was dispersed from a top or dispenser member on the container, and where a wick allowed the scented fluid to move from the interior of the container to the container top for dispersal or emanation. The wicks of certain types, such as those of U.S. Pat. No. 6,537,061, required heat or burning of a combustible material, usually a scented fluid, for the aromatic fragrance to be dispersed. If the dispenser were left unattended, the source of the heat or burning represented a fire or safety hazard. Other dispensers which were used without heat to disperse an aromatic fragrance required that the wick be exposed to the air. However, the exposed wick was visible and did not present an aesthetically pleasant appearance.

Other forms of vapor dispensers which attempted to avoid the problems of wicks were of specialized construction. Some, such as in U.S. Pat. No. 6,699,432 used a specially configured emanator plate or disk atop a fragrance container. The plate or disk configuration was limited and governed by functionality, and was of a form configured to accumulate an open, exposed pool of the fragrant fluid. The fluid was fed or supplied as a result of thermal expansion due to ambient temperature increases during the day.

Other forms of vaporized dispensers relied on different types of materials for the emanator, such as U.S. Pat. No. 5,725,152 (filter paper); U.S. Pat. No. 2,283,028 (cedar wood or some like wood); U.S. Pat. No. 4,413,779 (rigid porous polyethylene material); U.S. Pat. No. 4,915,301 (membrane with a substrate of a non-woven polyester sheet and coating layer of a microporous polymer); U.S. Pat. No. 1,123,036 (mixture of plaster-of-Paris, alum and sand); and U.S. Pat. No. 4,489,286 (totally synthetic, microporous synthetic plastic sheet material composed primarily of polyethylene). So far as is known, these vapor dispensers have not achieved acceptance or wide utilization in the trade.

### SUMMARY OF THE INVENTION

Briefly, the present invention provides a new and improved dispenser container for a vaporous scented aromatic fluid. The dispenser container according to the present invention includes a holder for receiving the fluid, with a cap member being mounted on the holder to close an upper portion of the holder. The cap member of the dispenser container is formed of a clay material which has been fired to form a bisque material. The cap member is non-pliable and porous and allows passage of vapors from the fluid in the fluid holder into the atmosphere in the vicinity of the container. A wick mounted with the cap member extends into an interior portion of the fluid holder. The wick transports the fluid from the interior portion of the fluid holder to the cap member so that vapors from the fluid pass through the bisque material of the cap member to form an aromatic or scented region in the vicinity of the dispenser container.

To better understand the characteristics of the invention, the description herein is attached, as an integral part of the same, with drawings to illustrate, but not limited to that, described as follows.

### BRIEF DESCRIPTION OF THE DRAWINGS

A better understanding of the present invention can be obtained when the detailed description set forth below is reviewed in conjunction with the accompanying drawings, in which:

FIG. 1 is an exploded front elevation view of an aromatic dispenser container according to the present invention.

FIG. 2 is an exploded front elevation view of another form of an aromatic dispenser container according to the present invention.

FIG. 3 is a front elevation view of an assembled aromatic dispenser container according to FIG. 1.

FIG. 4 is an enlarged view, taken partly in cross-section, of a portion of the aromatic dispenser container of FIG. 3.

FIG. 4A is an enlarged view, with portions of the structure partially removed, of an alternate structure for connecting a cap member and fluid holder of an aromatic dispenser container according to the present invention.

FIG. 5 is an enlarged view, taken in vertical cross-section, of the structure of the aromatic dispenser container shown in FIG. 4.

FIG. 6 is a front elevation view of an aromatic dispenser container according to the present invention having a different form of cap member than that shown in FIG. 1.

To better understand the invention, a detailed description of an illustrative example, as shown in the drawings for illustrative but not limiting purposes, is included as part of the description herein.

### DETAILED DESCRIPTION OF THE PREFERRED EMBODIMENT

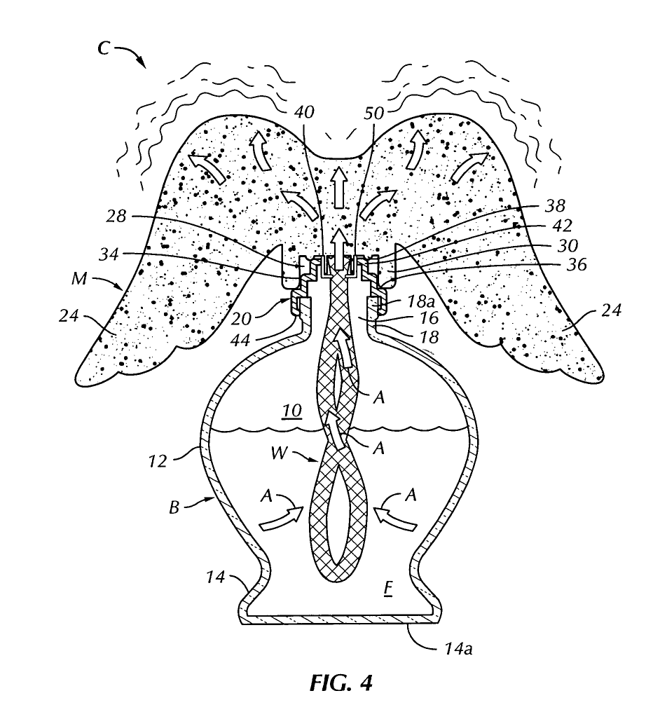

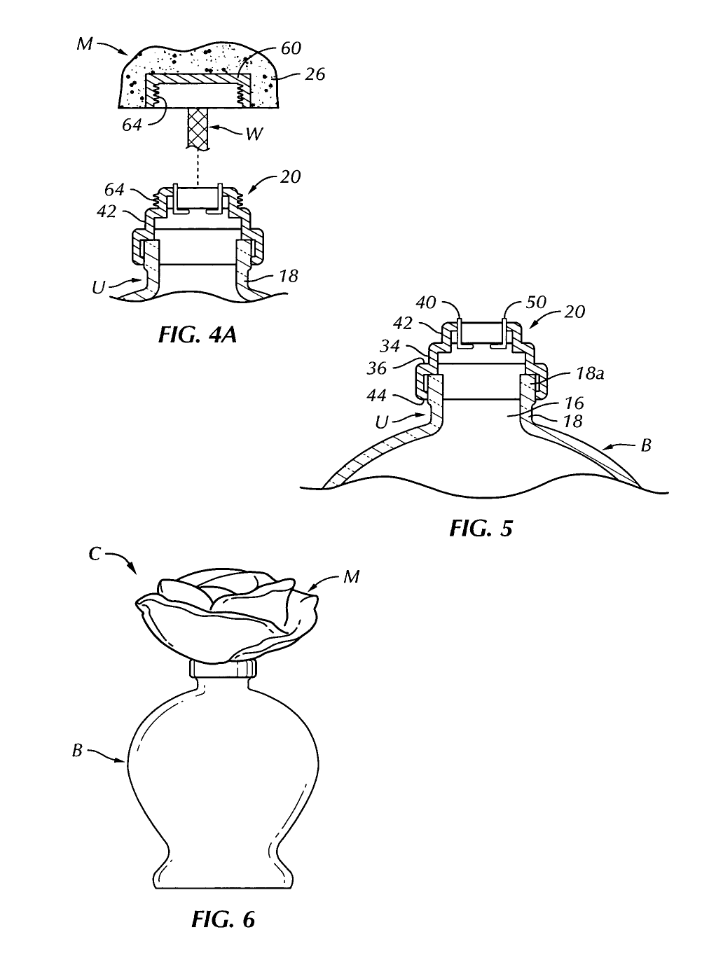

In the drawings, the letter C designates generally an aromatic dispenser container according to the present invention. The aromatic dispenser container C provides a new and improved dispenser container for a vaporous scented aromatic fluid as shown at F (FIG. 4). The aromatic dispenser container C according to the present invention includes a holder bottle or vessel B for receiving the fluid F, with a cap member M being mounted on the holder B to close portion or neck U of the holder B.

The cap member M of the aromatic dispenser container C is formed of a porous clay material, as will be set forth below, which has been fired to form a bisque material. The cap member M is non-pliable and porous and allows passage of vapors from the fluid F in the fluid holder B into the atmosphere in the vicinity of the container C.

The wick W is mounted with the cap member M and extends into an interior portion 10 of the fluid holder B. The wick W transports the fluid F from the interior portion 10 of the fluid holder B to the cap member M so that vapors from the fluid F pass through the bisque material of the cap member M to form an aromatic or scented region in the vicinity of the dispenser container C.

The aromatic dispenser container C of the present invention dispenses a scent or aroma, based on vapors originating from the fluid F held in the fluid holder B. The fluid F need not be flammable, and no flame or heat source is required for the vapors to emanate from the container C, as will be set forth. Examples of suitable fluids which may be used in connection with the aromatic dispenser container C according to the present invention are liquids of the type known as essential oils. These liquids are volatile oils that occur in plants and are characterized

odors or flavors. For the purpose of the present invention, oils of pleasant fragrance or otherwise desirable for health, therapy, aromatic or other reasons are used. In some situations, other scented or fragrant oils, even if combustible, may be used. In such cases, however, no flame or heat source is needed, as noted above.

The fluid F which vaporizes to give rise to the scent or aroma is contained within the holder B, which may be a bottle, jar or other type of fluid holder or container, typically of some decorative configuration. Considering the holder B more in detail, a fluid holder portion 12 of the holder B is supported by a base portion or pedestal 14 for positioning the holder B in a suitable location in a room or other area in which it is desired to provide the scent or aroma. The holder B is also provided with a top opening or neck 16 adjacent an upwardly extending wall 18 formed with the fluid holder portion 12. The fluid holder portion 12 may be formed of any suitable fluid retaining material, such as glass, metal or the like, preferably a material substantially impervious to or at least resistant to vapor passage through its walls or body portions.

The fluid holder portion 12 of the holder B may be of a suitable size and fluid holding capacity, and an example of a small fluid holder would be one of from two to eight fluid ounces capacity. The fluid holder portion 12 may be formed or configured into a wide variety of visually appealing or decorative shapes such as a sphere or globe as shown in the drawings, a cube, box, or other geometrical shape than a sphere or globe. The fluid holder portion 12 may be also be configured to resemble objects or things appearing in nature, figures, figurines, or art objects of various sizes and configurations. The fluid holder portion can accordingly take the form of a variety of ornamental shapes or forms, subject to affording sufficient space within the interior to hold a suitable amount of fluid F for purposes of forming adequate vapor or scent.

The base or pedestal 14 of the fluid holder B is typically integrally formed with the fluid holder portion 12 along its lower or bottom portions and serves to provide a stable base or rest for the holder B on the surface or area where it is positioned. The base 14 is of a size adequate for stability and is configured to either supplement or to blend in with the appearance or décor of the fluid holder portion 12. The base or pedestal 14 thus has either a flat lower surface 14a as shown or a suitable number of support legs for stability purposes. It should be understood that the base or pedestal member may be separately formed as a different structural component of the container C from the fluid holder portion 12, if desired.

The top opening neck 16 of the fluid holder B is provided with a top or connector ring member 20 which is attached to form a closed container for the scented fluid F. The connector ring member 20 is fitted onto an upper rim 18a of the wall 18 above the fluid holder portion 12. The connector ring member 20 is preferably snugly fitted onto the wall 18 to reduce fluid or vapor leakage between the ring member 20 and the fluid holder portion 12 at their juncture. The connector ring member 20 has a central opening or port 22 through which the wick W may be inserted into the fluid F in the container C.

The cap member M is formed of a porous, clay-type material of the type used in forming porcelain articles. Suitable examples of a porous, clay-type material which may be used according to the present invention are those known as ceramic, plaster, porcelain or gypsum materials, or mixtures of such materials. While still in the form of what is known as a wet clay, before first firing the clay material for the cap member M is shaped in a casting mold or otherwise to form it any one of a virtually unlimited variety of ornamental or aesthetically pleasing shapes or figures to give the dispenser C a decorative appearance.

The particular ornamental shape or appearance for cap member M is, as has been noted, any one of a wide variety of visually appealing or decorative shapes. Examples may be found from objects, things, figures, forms found in nature, custom designed or arbitrary imagined or designed figures, figurines, or art objects of various sizes and configurations, or other forms which might occur to a sculptor's or designer's imagination. The variety of shapes and designs for the cap member M is even greater than in the case of container C, since there is no need to provide interior space in the cap member M for containing a supply or reservoir of fluid to be dispensed.

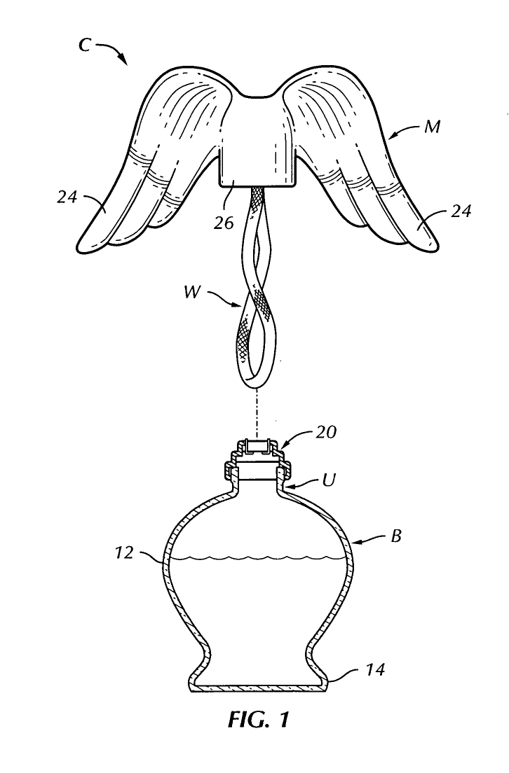

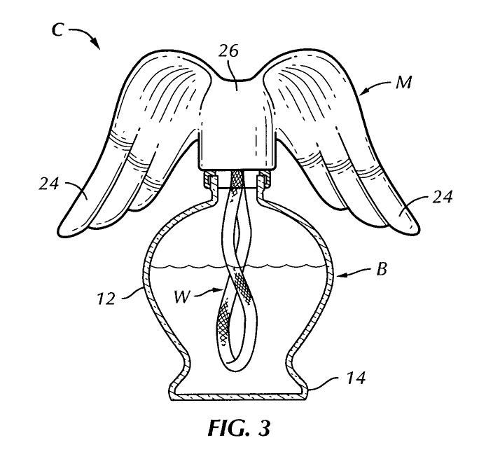

In FIGS. 1-4 of the drawings, an illustrative embodiment for a cap member M is in shown in the form of a pair of angel or peace wings 24 extending outwardly from opposite sides of a rounded cylindrical pedestal or base 26 are shown by way of an illustrative embodiment. In FIG. 6, the cap member M as an example of another aesthetic or ornamental choice takes the form of a rose blossom 25. The particular shape and design of the cap member M is a matter of aesthetic or ornamental choice, as noted above. The cylindrical base 26 of the cap member M in either example form (FIGS. 1-4 or FIG. 6) has an upwardly extending opening or mounting socket 28 (FIG. 5) formed therein adjacent a lower cylindrical side wall 30. The mounting socket 28 so formed provides a space into which the connector ring 20 is fitted. The inner diameter of the cylindrical side wall 30 is sized to permit the cap member M to fit over and snugly onto an outer surface 34 in an intermediate section 36 of the connector ring 20. The rose blossom 25 shown in FIG. 6 has a like base interior structure to that shown in FIG. 5 and described above.

The cap member M has a raised circular positioning rim or lip member 38 formed extending downwardly from an upper inner wall 40 above the mounting socket 28 to provide contact with an upper section 42 of the connector ring 20. The connector ring 20 also includes a lower section 44 having an inner diameter sized to snugly fit over the top neck wall 18 of the fluid holder B.

If desired, the cap member M may as shown in FIG. 4A be provided with an internal threaded cup wall or an insert cup 60 of some suitable synthetic resin or metal with an internal threaded wall 62. The threaded surfaces 62 serve to engage and receive a threaded outer surface 64 formed on either surface 34 or 42 of container ring 20. The threaded connection shown in FIG. 4A is provided for use where it is desired to protect against the cap member M becoming dislodged from the fluid holder B. In this manner, possible spillage of fluid from the open top of the fluid holder B is prevented.

With either the fitted or threaded connection between cap member M and fluid holder B, vapors from the fluid F are confined in the fluid holder B except to the extent such vapors are allowed pass through the bisque material of the cap member M to form an aromatic or scented region in the vicinity of the dispenser container C.

The clay material of the cap member M after being molded or otherwise shaped into a suitable ornamental form is allowed to dry. After drying has been accomplished, the cap member M is subject during a first firing known as bisque, or biscuit, firing. Bisque firing gives the material of the cap member M greater strength and resistance to breakage than that of greenware. It can thus be handled with less risk of breakage, although some care is still required. With the present invention it has been found that the material of the cap member M after bisque or biscuit firing, and thus named bisque material according to the present invention, still exhibits adequate porosity or permeability so that vapors from a fluid of the type described above may pass from the interior of fluid holder B into the atmosphere in the vicinity of the container C.

The bisque material of the cap member M is still permeable and porous enough to allow the vapors from the fluid in the container to permeate from the container bottle B through the bisque material and into the nearby region. The fluid vapors fluid pass through the bisque material of the cap member M to form an aromatic or scented region in the vicinity of the dispenser container. The aromatic or scented region is achieved without need for heat or burning of a combustible scented fluid in order for the aromatic fragrance to be dispersed.

The wick W which transports the fluid F from the interior portion 10 of the fluid holder B to the cap member M is mounted with the upper inner wall 40 of the cap member M. When the cap member M is mounted on the connector ring member 20, the wick W extends into the fluid in the interior portion 10 of the fluid holder B. The wick W transports the aromatic vaporous fluid F from the interior portion 10 to the cap member M by wicking or capillary action so that the vapors may pass through the bisque material for dispersal.

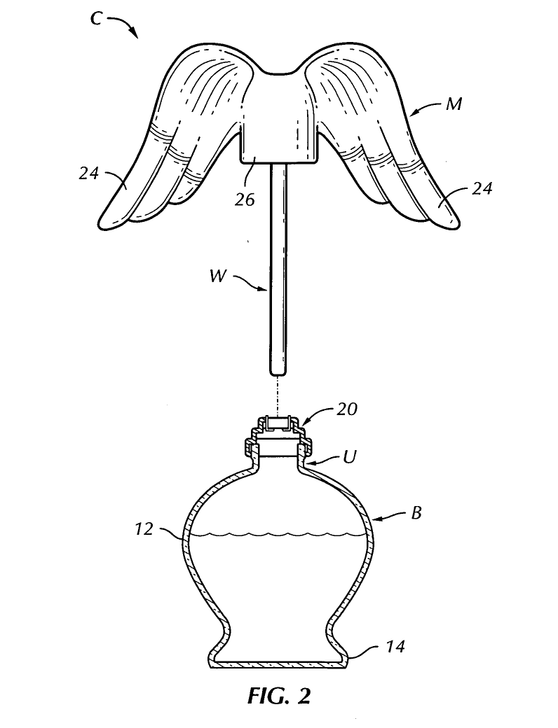

The wick W may be mounted and assembled for use with the cap member M in several ways with the present invention. The wick W may for example be mounted by being integrally formed as shown at 46 (FIG. 2) in same molding process with the cap member M as an integral unit. The wick W and cap member M are thus integrally formed as a single item of bisque material, if desired.

The wick W may also be a separate item 48 (as shown in FIGS. 1, 3 and 4) of some materials other than bisque, but a material possessing a wicking or capillary capability. The wick W thus serves to provide transport of the fluid from the interior of the fluid holder B to the cap member B for aromatic vapor dispersion. For example, the material 48 for the wick W may be formed of a suitable fabric, such as cotton, synthetic fibers or mixtures or blends thereof. When the wick W is of fabric material 48, or other fabric material separate from and not integral with the cap member M, the wick W and cap member M are mounted together a suitable fastener 50, such as a clip, snap, clasp or the like being used for attachment purposes.

The foregoing disclosure and description of the invention are illustrative and explanatory thereof, and various changes in materials, sizes, and configurations, as well as methods of use and particular pants garments may be made without departing from the spirit of the invention, the scope of which is defined in the following claims.

While the invention has been described herein with respect to certain embodiments, it should be understood by those that are skilled in the art that it is not so limited. The invention is susceptible of various modifications and changes without departing from the scope of the claims.

What is claimed is:

1. A dispenser container for a vaporous scented aromatic fluid, comprising:
a holder for receiving the vaporous fluid;
a cap member mounted on the holder and closing an upper portion of the holder;
the cap member being formed of a porous clay material which has been fired to form a bisque material which is non-pliable and porous for passage of vapors therethrough into the atmosphere in the vicinity of the container; and
a wick mounted with the cap member and extending into an interior portion of the holder, the wick transporting the fluid from the interior portion of the holder to the cap member.

2. The dispenser container of claim 1, wherein the wick is formed of a like material to the cap member.

3. The dispenser container of claim 2, wherein the wick is integrally formed as a unit with the cap member.

4. The dispenser container of claim 1, wherein the wick is formed of fabric capable of transporting the fluid by wicking action.

5. The dispenser container of claim 4, wherein the fabric comprises cotton.

6. The dispenser container of claim 4, wherein the fabric comprises a synthetic fiber.

7. The dispenser container of claim 4, wherein the fabric comprises a blend of cotton and synthetic fibers.

8. The dispenser container of claim 1, wherein the wick is attached to the cap member.

9. The dispenser container of claim 5, wherein the wick is formed of fabric capable of transporting the fluid by wicking action.

10. The dispenser of claim 9, wherein the fabric comprises cotton.

11. The dispenser of claim 9, wherein the fabric comprises a synthetic fiber.

12. The dispenser of claim 9, wherein the fabric comprises a blend of cotton and synthetic fibers.

13. The dispenser of claim 1, wherein the cap member is fitted onto the holder.

14. The dispenser of claim 1, wherein the cap member is mounted by a threaded connection onto the holder.
